(19) United States
(12) Patent Application Publication
(10) Pub. No.: US 2026/0344987 A1
(43) Pub. Date: Nov. 5, 2026

(54) ARTICULATABLE MEDICAL SCOPE WITH INTEGRATED IRRIGATION CONTROL

(71) Applicant: Verathon Inc., Bothell, WA (US)

(72) Inventors: Alex B. Antonio, Burnaby (CA); Gilbert Wong, Burnaby (CA); Fabio Bollinger, Surrey (CA)

(73) Assignee: Verathon Inc., Bothell, WA (US)

(21) Appl. No.: 19/665,047

(22) Filed: May 1, 2026

Related U.S. Application Data
(60) Provisional application No. 63/798,133, filed on May 1, 2025.

(51) Int. Cl. A61B 1/015; A61B 1/005; A61B 1/018; A61B 1/05; A61B 1/307

(52) U.S. Cl. CPC A61B 1/015; A61B 1/0052; A61B 1/018; A61B 1/05; A61B 1/307

(57) ABSTRACT

A medical scope device includes a handle and a shaft projecting from the handle. The shaft includes a proximal end coupled to the handle and a distal end. A working channel extends through the shaft. A flexible tip is coupled to the distal end of the shaft. The handle includes a flexible tip control mechanism operatively coupled to the flexible tip and a control lever coupled to the flexible tip control mechanism. The control lever extends from the handle in a first location and manipulation of the control lever causes deflection of the flexible tip. The handle further includes an irrigation control system for controlling a flow of irrigation fluid from an irrigation fluid source to the working channel. The irrigation control system includes an irrigation control lever that extends from the handle in a second location. The first location is substantially opposite the second location.

### CROSS-REFERENCE TO RELATED APPLICATIONS

This application claims benefit under 35 U.S.C. § 119(e) to U.S. Provisional Application Serial No. 63/798,133, filed May 1, 2025, entitled “ARTICULATABLE MEDICAL SCOPE WITH INTEGRATED IRRIGATION CONTROL,” the entire disclosure of which is incorporated by reference herein.

### BACKGROUND

An endoscope refers to a medical device that allows remote examination of the interior of a patient’s body. Endoscopes may be used for a variety of diagnostic and treatment procedures relating, for example, to the gastrointestinal, respiratory, reproductive, and urinary tract systems.

### SUMMARY

In various embodiments, a medical scope device is disclosed. The medical scope device includes a handle, a shaft projecting from the handle, a working channel extending through the shaft, and a flexible tip. The shaft includes a proximal end coupled to the handle and a distal end. The flexible tip is coupled to the distal end of the shaft. The handle includes a flexible tip control mechanism operatively coupled to the flexible tip, a control lever coupled to the flexible tip control mechanism, and an irrigation control system for controlling a flow of irrigation fluid from an irrigation fluid source to the working channel. The control lever extends from the handle in a first location. Manipulation of the control lever causes deflection of the flexible tip and the irrigation control system includes an irrigation control lever that extends from the handle in a second location. The first location is substantially opposite to the second location.

In various embodiments, a cystoscope device is disclosed. The cystoscope device includes a handle, a shaft projecting from the handle, and a working channel extending through the shaft. The shaft includes a flexible tip. The handle includes a flexible tip control mechanism operatively coupled to the flexible tip, an irrigation fluid port for receiving a source of irrigation fluid, and an irrigation control system for controlling a flow of irrigation fluid from the irrigation fluid port to the working channel. The flexible tip control mechanism includes a flexible tip control lever that projects from the handle at a first location; and the irrigation control system includes an irrigation control lever that projects from the handle at a second location. The first location is opposite the second location.

### BRIEF DESCRIPTION OF THE DRAWINGS

The features and advantages of the present disclosure will be more fully disclosed in, or rendered obvious by the following detailed description of example embodiments, which are to be considered together with the accompanying drawings wherein like numbers refer to like parts and further wherein:

FIG. 1 is a diagram illustrating an endoscope system, in accordance with some embodiments;

FIG. 2A is an exploded front perspective view of the medical scope device of FIG. 1, in accordance with some embodiments;

FIG. 2B is a partial left side view of a handle portion of medical scope device of FIG. 2A, in accordance with some embodiments;

FIG. 2C is a right side view of the left shell of FIG. 2A, in accordance with some embodiments;

FIGS. 3A and 3B are left side and right side views, respectively, of the control wheel and control lever of FIGS. 2A and 2B, in accordance with some embodiments;

FIGS. 3C and 3D are front and rear left isometric views, respectively, of the control mechanism of FIG. 2A, in accordance with some embodiments;

FIG. 4A is an exploded left side view of the access port assembly, irrigation control assembly, and irrigation port of FIG. 2A, in accordance with some embodiments;

FIG. 4B is a cross-sectional view of the access port assembly of FIG. 4A, in accordance with some embodiments;

FIG. 4C is an exploded rear isometric view of the access port assembly of FIG. 4A, in accordance with some embodiments;

FIG. 5A is a front view of the irrigation control assembly of FIG. 4A, in accordance with some embodiments;

FIG. 5B is a front left isometric view of the irrigation control assembly of FIG. 4A, in accordance with some embodiments;

FIG. 5C is an exploded rear left isometric view of the irrigation control assembly of FIG. 4A, in accordance with some embodiments;

FIG. 5D is a cross-sectional view of the irrigation control assembly of FIG. 5A, taken along the line B-B in FIG. 5A, in accordance with some embodiments; and

FIG. 5E is a cross-sectional view of the irrigation control assembly of FIG. 5D in a closed configuration, in accordance with some embodiments.

### DETAILED DESCRIPTION

Unless otherwise specifically defined herein, all terms are to be given their broadest reasonable interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “above” versus “below,” “inwardly” versus “outwardly,” “longitudinal” versus “lateral,” and the like are to be interpreted relative to one another or relative to an axis of elongation, or an axis or center of rotation, as appropriate. Terms concerning attachments, coupling, and the like, such as “connected” and “interconnected,” refer to a relationship wherein structures are secured or attached to one another either directly or indirectly through intervening structures. The terms “operatively connected,” “operably connected,” and the like are such attachments, couplings, or connections that allow the pertinent structures to operate as intended by virtue of that relationship.

In various embodiments, a medical scope system enables examination and irrigation of an internal cavity of a patient. The medical scope system includes a video medical scope device that includes a flexible tip that is controlled by manipulating a control lever in a handle of the endoscope device. In some embodiments, the video medical scope device includes an integrated irrigation control system for allowing users of the scope device to accurately and ergonomically control the flow of irrigation fluid through a working channel. In some embodiments, the irrigation control system is positioned oppositely from the control lever, so as to be utilized in conjunction with the flexible tip manipulation to target specific regions for irrigation within the patient.

Embodiments of the medical scope system described herein include both single-use (i.e., disposable) and reusable scope devices that may include image capturing and lighting elements, as well as additional features. During and after insertion of the medical scope device, images obtained from the image capturing elements may be conveyed to a video monitor viewable by the device user via a data cable.

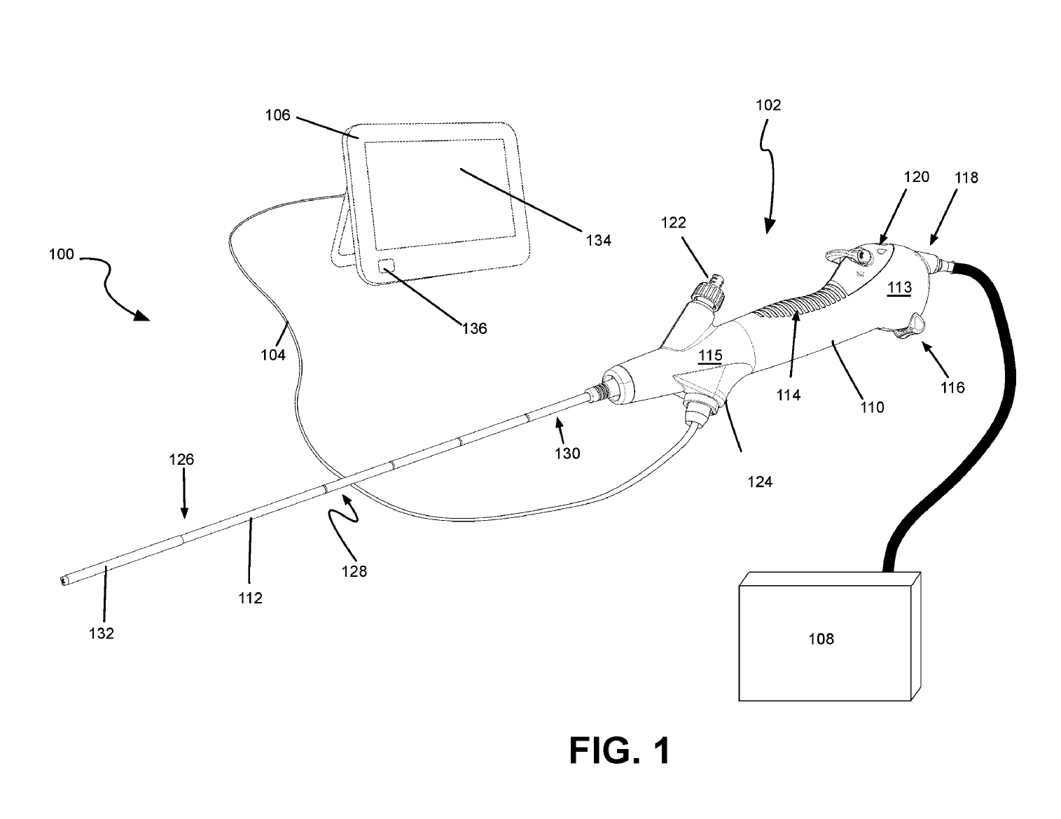

FIG. 1 illustrates a medical scope system 100, in accordance with some embodiments. Medical scope system 100 includes a scope device 102, a data cable 104, a video monitor 106, and an irrigation fluid source 108. Scope device 102 may include any of a variety of types of scope devices, such as endoscopes, borescopes, bronchoscopes, cystoscopes, ureteroscopes, gastroscopes, etc.

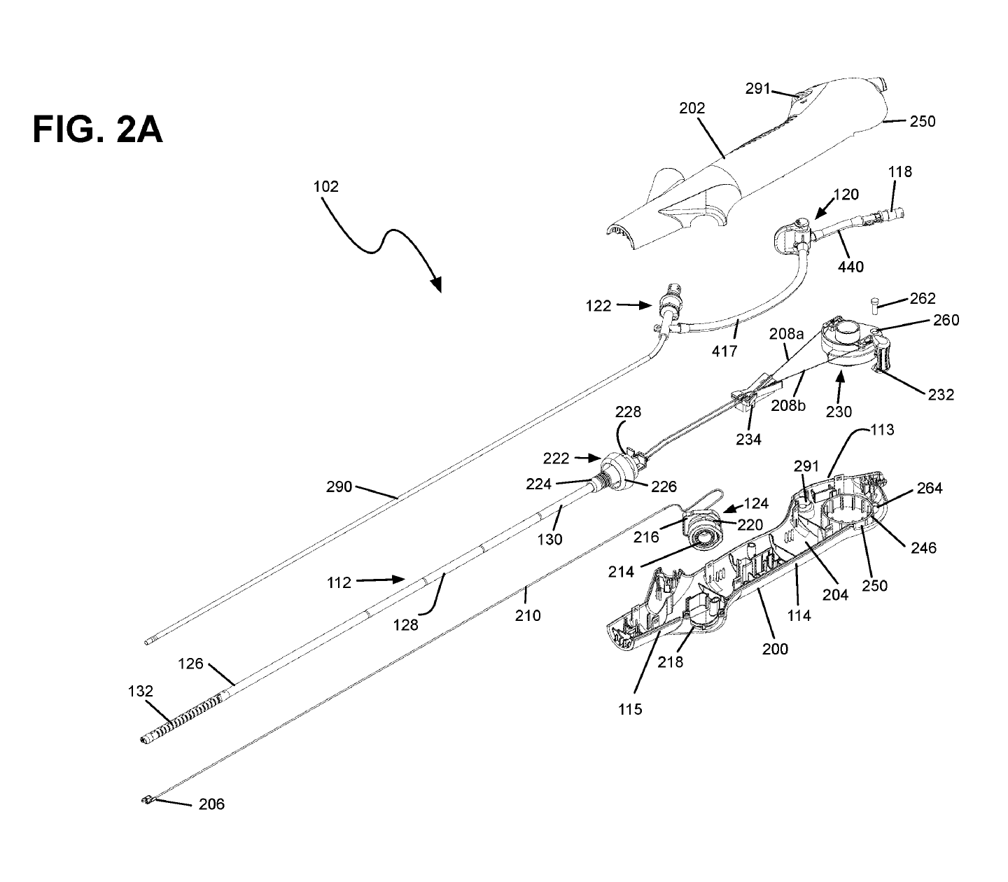

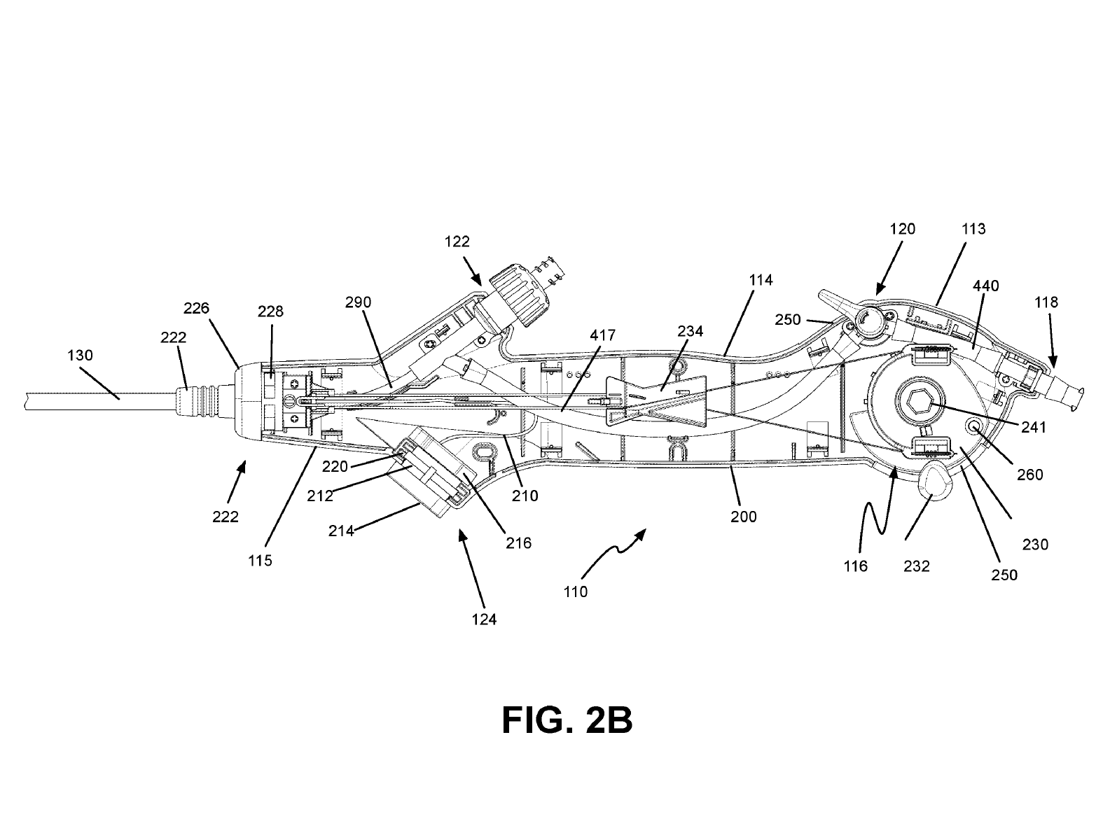

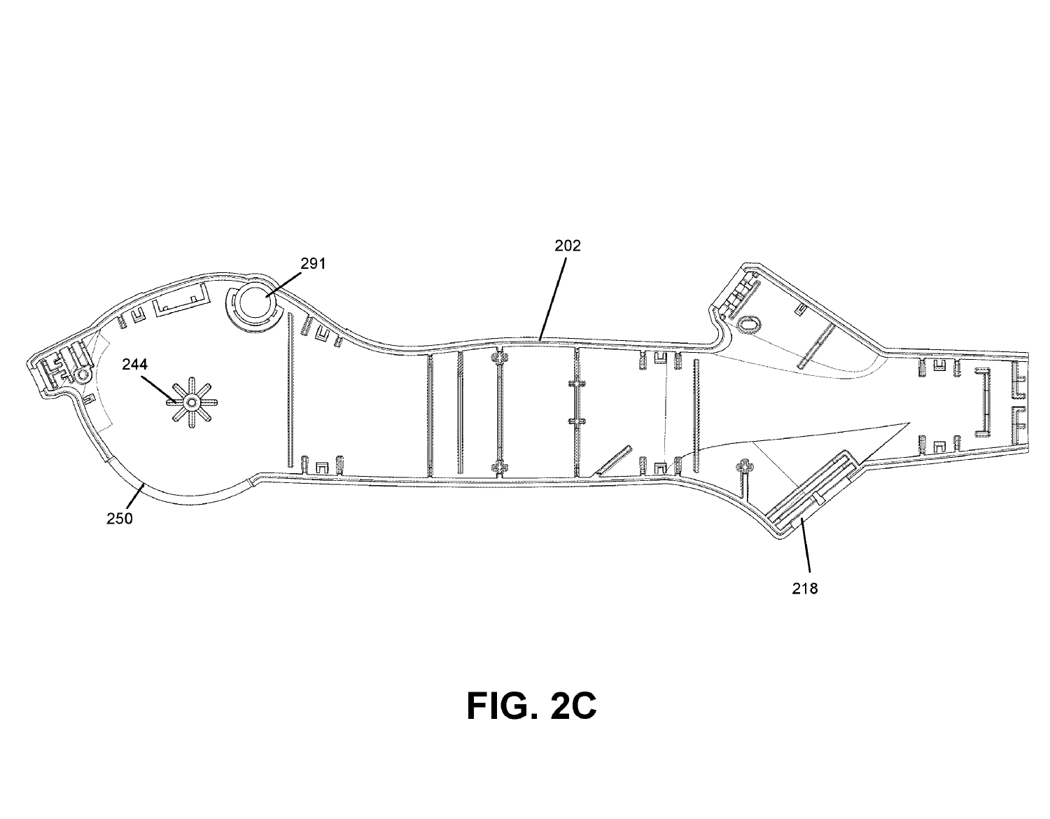

As shown in FIG. 1, scope device 102 includes a handle 110 and a shaft 112. Shaft 112 couples with and projects longitudinally from handle 110. As described in additional detail below, handle 110 may be formed of two similar sized halves, referred to as a right shell 200 and a left shell 202, which snap or otherwise connect together along a longitudinal center line of handle 110 to form an inner cavity 204 (see, e.g., FIG. 2A). When assembled, handle 110 includes, among other things, a control portion 113, a grip portion 114, and a distal portion 115. Control portion 113 of handle 110 includes a flexible tip control mechanism 116, an irrigation port 118, and an irrigation control assembly 120. In some embodiments, control portion 113 may include an enlarged configuration relative to grip portion 114. Distal portion 115 of handle 110 includes an access port assembly 122 and a data interface 124. Shaft 112 includes a distal end 126, an intermediate portion 128, and a proximal end 130 relative to handle 110. Distal end 126 includes a flexible tip 132. Dimensions of shaft 112 (e.g., length, outside diameter, and inside diameter) may vary based on an intended use of scope device 102, such as intended procedures, patient size, etc.

During use, flexible tip 132 of scope device 102 is introduced into a body cavity being inspected. In some embodiments, imaging module 206 (see FIG. 2A) is provided at distal end 126 of shaft 112 so as to capture and transmit images of the distal end 126 and corresponding patient anatomy to video monitor 106 via data cable 104. Articulation of flexible tip 132 may be enabled by one or more pull wires 208 (see FIG. 2A) coupled between flexible tip 132 and flexible tip control mechanism 116. Manipulation of flexible tip control mechanism 116 may cause flexible tip 132 to flex or bend in at least one plane.

In some embodiments, video monitor 106 provides power to and initiate image capture from scope device 102 via data cable 104. For example, as shown in FIG. 1, video monitor 106 may include a display 134, and a control interface 136. Practitioners (e.g., medical personnel) may interface with video monitor 106 during use to initiate image capture, freeze a particular frame, or adjust certain settings. Although not shown in the Figures, video monitor 106 may also include a data cable interface for receiving an end of data cable 104, a battery or other power source, and a remote monitor interface for enabling the view of display 134 to be transmitted to one or more other display monitors.

In some embodiments, shaft 112 is formed of a number of discrete components. For example, proximal and intermediate portions 130/128 of shaft 112 may be formed of a braided, semi-rigid polymer material having a single lumen therethrough, sized to accommodate the internal components described below, such as a working channel 290 (see FIG. 2A). Flexible tip 132, in contrast, may be formed of an injection molded material that includes a plurality of distinct lumens and a series of articulation slits on its outer periphery, although other configurations of flexible tip 132 may be used.

FIG. 2A illustrates an exploded front perspective view of medical scope device 102, in accordance with some embodiments. FIG. 2B is a left side view of a handle portion of medical scope device 102 in which a left shell 202 has been removed to depict the components inside the handle. FIG. 2C is a right side view of left shell 202.

As shown in FIGS. 2A and 2C, each of right shell 200 and left shell 202 include various integrally formed features that project into inner cavity 204 configured to support and secure the internal components of handle 110. For example, each of right shell 200 and left shell 202 include one or more clips, stand-offs, support members, and/or bosses configured to support such components and/or to engage corresponding features on the opposing shell. Furthermore, the various integrally formed features are formed so as not to conflict with the travel of pull wires 208 during operation of scope device 102.

As briefly described above, scope device 102 includes an imaging module 206 within flexible tip 132. In some embodiments, imaging module 206 includes a camera module and a light source module. Consistent with embodiments described herein, the components of imaging module 206 may be potted within flexible tip 132 using a curable adhesive, such as an ultraviolet light curable adhesive, after assembly.

Furthermore, in some embodiments, one or more portions of imaging module 206 may be formed as part of a circuit board assembly, such as a printed circuit board assembly (PCBA), flexible printed circuit board assembly (FPCBA), or rigid flexible printed circuit board assembly (RFPCBA) (not shown). In one embodiment, the PCBA (or FPCBA/RFPCBA) may be configured to couple imaging module 206 to data interface 124 mounted in handle 110 via electrical wires 210 that extend the length of medical scope device 102. In alternative embodiments, imaging module 206 may be coupled directly to wires 210 and may not be integrated with or coupled to a PCBA. In yet another embodiment (not shown), at least a portion of imaging module 206 may be integrated within or provided as an additional long, flexible PCBA that extends directly from the imaging module 206 to data interface 124, without the need for discrete electrical wires, in accordance with some embodiments.

In some embodiments, data interface 124 provides an interface between imaging module 206 and video monitor 106. As shown in FIG. 2A, data interface 124 may include an interface housing 212, an interface component 214, and a printed circuit board assembly (PCBA) 216. Interface housing 212 is received within a data interface opening 218 jointly formed in right shell 200 and left shell 202 of handle 110. Interface housing 212 includes engagement components 220 (e.g., channels, grooves, slots, etc.) formed on an outer surface thereof for facilitating secure retainment of interface housing 212 within data interface opening 218.

In some embodiments, interface component 214 is secured within housing 212 and coupled to PCBA 216. As shown in FIG. 2A, interface component 214 may include a multi-pin, magnetic configuration. In other embodiments, different connector technologies may be used, such as high-definition multimedia interface (HDMI) or universal serial bus, type C (USB type C) connectors.

In some embodiments, proximal end 130 of shaft 112 includes a strain relief and tube engagement portion 222. Strain relief and tube engagement portion 222 may include an arrangement of a generally concentric interior strain relief component 224, a tube engagement component 226 joined at a portion (not shown) proximal to handle 110, and a handle interface portion 228.

Interior strain relief component 224 may be secured to proximal end 130 of shaft 112 to support the interface between shaft 112 and handle 110 and prevent breakage or removal of shaft 112 from handle 110. Tube engagement component 226 may be sized to receive and frictionally engage an outer device tube (not shown) received over shaft 112 for subsequent deployment into the patient’s body. Accordingly, tube engagement component 226 may include different sizes or combinations of sizes (e.g., its inside diameter) consistent with a device tube to be deployed.

Regardless of size or relative size, in each embodiment of strain relief and tube engagement portion 222, interior strain relief component 224 may be sized to receive proximal end 130 of shaft 112. During assembly of scope device 102, proximal end 130 may be secured, e.g., via adhesive, overmolded, interference fit, etc. to interior strain relief component 224. As described briefly above, tube engagement component 226 may be appropriately sized to receive an outside surface of a device tube. The outside diameter of interior strain relief component 224 may be sized smaller than the inner surface of a suitable device tube, so that only tube engagement component 226 engages the device tube.

In some embodiments, inner surface of tube engagement component 226 may include engagement features, such as ribs, detents, bumps, etc. (not shown) to aid in releasably engaging an outer diameter of a device tube. Furthermore, in some embodiments, a forward edge of tube engagement component 226 may be chamfered so as to more easily receive a device tube slid along shaft 112.

Handle interface portion 228 of strain relief and tube engagement portion 222 may extend proximally from interior strain relief component 224 and tube engagement component 226 and may include various features configured to engage corresponding features of right shell 200 and left shell 202 to secure strain relief and tube engagement portion 222 within handle 110. For example, handle interface portion 228 may include slots for receiving corresponding positioning members in right shell 200 and left shell 202 (positioning member in left shell 202 is shown in FIG. 2C).

In some embodiments, at least a portion of strain relief and tube engagement portion 222 may be formed of a resilient or semi-rigid material, such as a polymer or rubber, suitable for frictionally engaging proximal end 130 of shaft 112 and for engaging and retaining a device tube in an engagement position during initial use of scope device 102. In some embodiments, such material may effectively seal the distal end of handle 110 and prevent materials from entering cavity 204 therein.

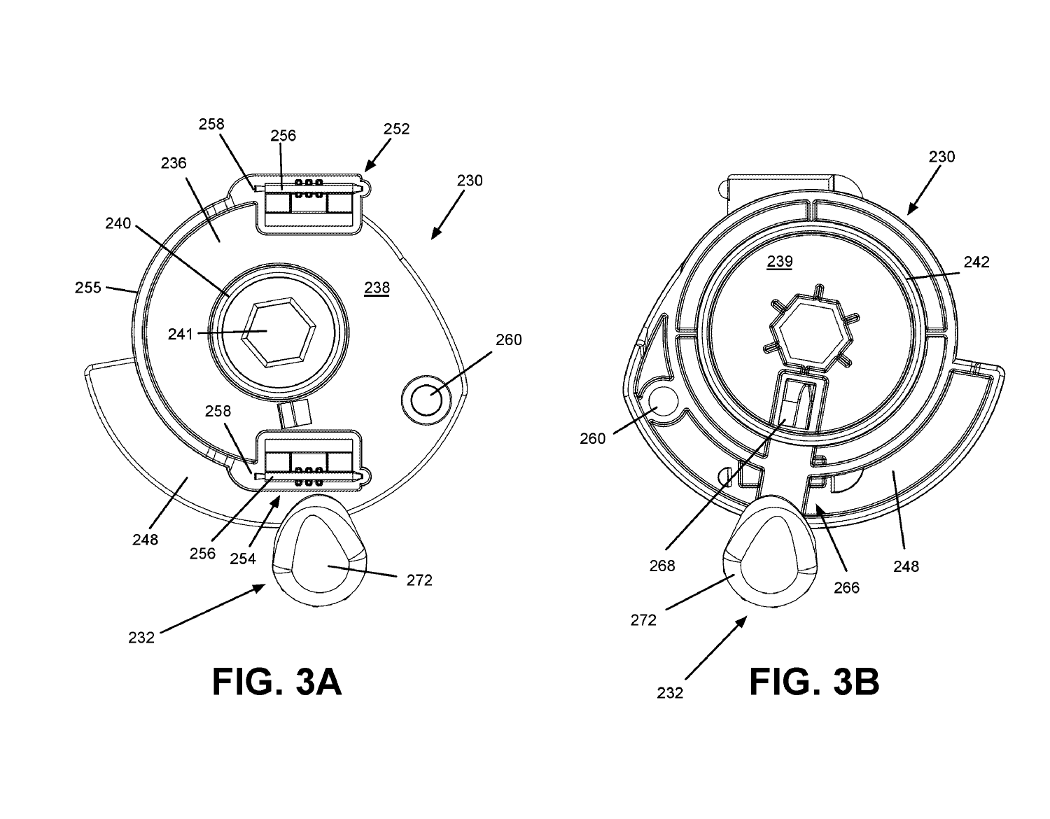

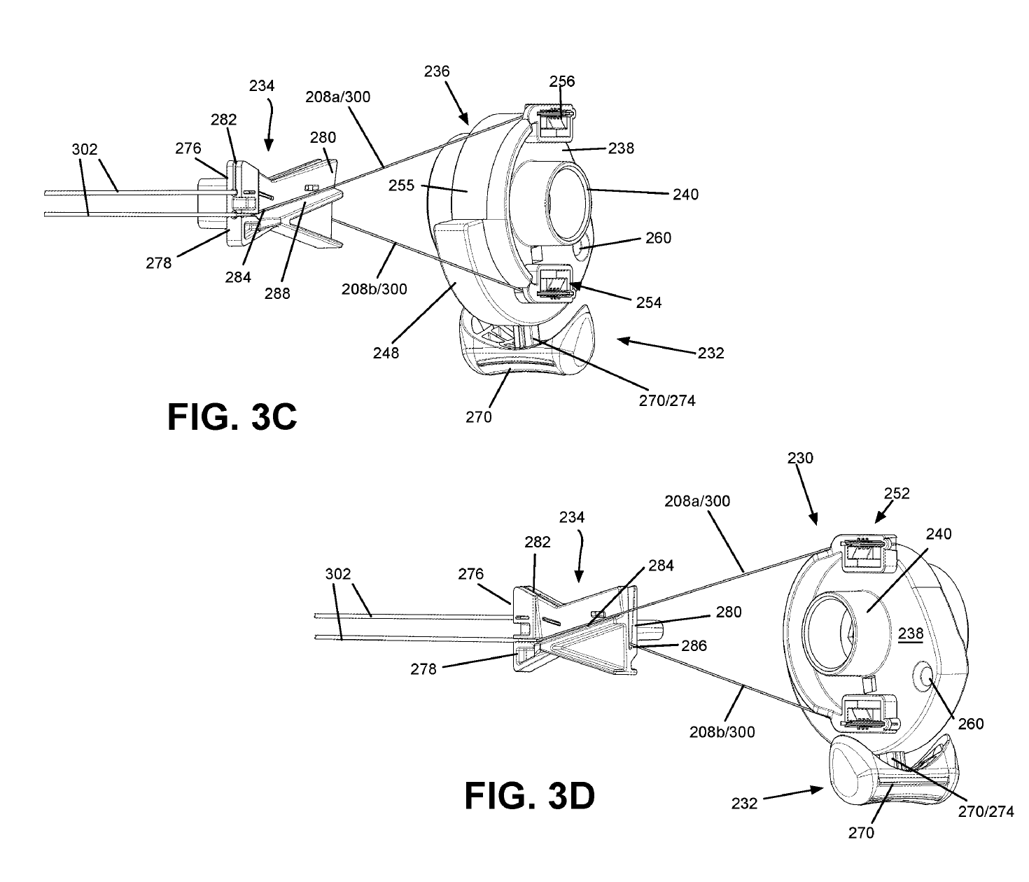

Turning to flexible tip control mechanism 116, in some embodiments, flexible tip control mechanism 116 may include a control wheel 230, a pair of pull wires 208a and 208b (referred to generally and collectively as “pull wires 208”), a control lever 232, and a compression coil stop 234. FIGS. 3A and 3B are left side and right side views, respectively, of control wheel 230 and control lever 232 consistent with embodiments described herein. FIGS. 3C and 3D are front and rear left isometric views, respectively, of flexible tip control mechanism 116.

As shown in FIGS. 2A-3D, control wheel 230 includes a generally disc-shaped body 236 rotatably mounted within inner cavity 204 of handle 110. As shown, disc-shaped body includes a left side 238 and a right side 239. In particular, in one embodiment, control wheel 230 includes a left shaft portion 240 having a centering element 241 therein that projects leftwardly from left side 238 of disc-shaped body 236 toward left shell 202 and a right shaft portion 242 that projects rightwardly from right side 239 of disc-shaped body 236 toward right shell 200. To accommodate rotational mounting within inner cavity 204, left shell 202 may include a corresponding left-side boss 244 (FIG. 2C) configured to engage centering element 241 and right shell 200 may include a right-side boss 246 (FIG. 2A) sized to receive right shaft portion 242. For example, left-side boss 244 may include a hexagonal or star-shaped feature that engages corresponding female hexagon shaped centering element 241 of left shaft portion 240 and an inside surface of right-side boss 246 may receive and engage an outside surface of right shaft portion 242, although alternative configurations may be made.

In some embodiments, disc-shaped body portion 236 includes a lobed or cam shaped configuration wherein at least an expanded portion 248 of an outer periphery of body portion 236 projects radially outward relative to a remainder of body portion 236. Accordingly, at least a portion of control wheel 230 is provided with differing outside diameters, with a first portion having a smaller outside diameter and a second portion having a larger outside diameter. As shown in FIG. 3A and 3C, expanded portion 248 may be configured to not engage or otherwise support pull wires 208 on an outer periphery thereof, but which is formed adjacent opening 250 (FIG. 2B) in handle 110 through which control lever 232 extends. As shown, handle 110 adjacent opening 250 is configured in a manner that substantially conforms to an outside configuration of expanded portion 248, such as an arced or circular configuration. By including expanded region 248 in this area of handle 110, any gap between control wheel 230 and opening 250 may be minimized, thereby preventing or minimizing the entry of foreign materials into inner cavity 204 which may affect operation of flexible tip control mechanism 116.

In some embodiments, body portion 236 of control wheel 230 includes a pair of pull wire troughs 252 and 254 for receiving and securing pull wires 208a and 208b, respectively, therein. As shown in FIGS. 3A and 3C, pull wire troughs 252/254 may be formed as parallel projections extending from a left side 238 of body portion 236 in an orientation generally perpendicular to an axis of rotation of control wheel 230.

In some embodiments, pull wire troughs 252 and 254 are formed on opposite sides of left shaft portion 240, such that rotation of control wheel 230 about left shaft portion 240 in a clockwise direction causes pull wire 208a to retract relative to shaft 112 and pull wire 208b to travel further into shaft 112, thus deflecting tip 132 downwardly (in FIG. 2A) relative to handle 110. Conversely, rotation of control wheel 230 about left shaft portion 240 in a counterclockwise direction causes pull wire 208a to travel further into shaft 112 and pull wire 208b to retract relative to shaft 112, thus deflecting tip 132 upwardly relative to handle 110. When control wheel 230 is rotated, the pull wire 208 being retracted engages a forward portion 255 of disc shaped body portion 236 adjacent its respective trough 252/254.

In some embodiments, each pull wire trough 252/254 includes a channel 256 therein for receiving its respective pull wire 208 upon assembly of scope device 102. Channels 256 may be sized to accommodate pull wires 208. In some embodiments, channels 256 may include a v-groove configuration for seating each pull wire 208 at a particular position relative to channel 256 (e.g., centered within the channel, etc.). Consistent with embodiments described herein, each pull wire trough 252/254 includes an enclosing portion 258 for covering at least a portion of the channels 256 within troughs 252/254. Such a configuration prevents pull wires secured within troughs from falling out of channel 256 and becoming disengaged from the outer periphery of control wheel 230 prior to or during use. In some embodiments, enclosing portion 258 may extend forwardly from troughs 252/254 to the outer periphery of control wheel 230 to prevent pull wires 208 from becoming disengaged from control wheel 230.

In some embodiments, body portion 236 of control wheel 230 may be provided with a tensioning pin aperture 260 for receiving a tensioning pin 262 (FIG. 2A) therethrough during assembly of scope device 102. As shown in FIGS. 2A and 2B, tensioning pin aperture 260 may align with tensioning pin boss 264 provided in right shell 200, so that tensioning pin 262 may be received into tensioning pin boss 264 through tensioning pin aperture 260 during assembly of scope device 102 to fix the rotation of control wheel 230 relative to right shell. When rotationally fixed, pull wires 208 may be received within channels 256 and their respective tensions may be adjusted to obtain a neutral setup, with pull wires 208a, 208b exhibiting equivalent tension. Once such a neutral setup is achieved, pull wires 208a, 208b may be secured within troughs 252/254, such as via a glue, or suitable clamping mechanism. In one embodiment, a self-curing adhesive or UV-curable glue may be applied to pull wires 208 within troughs 252/254 and allowed to cure or UV cured. At this point in the assembly, tensioning pin 262 may be removed, thus allowing control wheel 230 to rotate freely relative to handle 110.

In some embodiments, control wheel 230 further includes a control lever engagement portion 266. Control lever engagement portion 266 includes a radially inward slot 268 formed in a periphery of body portion 236 of control wheel 230 and configured to align with a corresponding opening 250 in handle 110. As shown in FIG. 2A, opening 250 in handle 110 is created by providing corresponding slots in right shell 200 and left shell 202. In some embodiments, radially inward slot 268 may include a clip element for allowing releasable engagement with shaft 274 of control lever 232. In other embodiments, control lever 232 may be secured within control level engagement portion 266 by other means, such as adhesive, a friction fit, etc.

Control lever 232 may include a generally T-shaped body 270 having a user engagement portion 272 and a shaft portion 274 that projects downwardly from engagement portion 272. As shown, user engagement portion 272 may be configured for easy forward/backward manipulation by a user’s thumb during operation of scope device 102. In some embodiments, user engagement portion 272 includes a curved lateral profile that generally mirrors an outer configuration of handle 110. Such a feature minimizes the likelihood that control lever 232 will get caught up on various environmental elements, such as clothing, equipment, wires/cables, etc. An outer surface of control lever 232, may include a friction surface, such as ribbed, grooved, or knurled surface. Such a configuration reduces the likelihood that a user’s thumb will slip off of control lever 232 during use. Shaft portion 274 of control lever 232 may include an external configuration that corresponds to slot 268 in control lever engagement portion 266, which may include a rectangular, cylindrical, or tapered configuration. As briefly described above, shaft portion 274 may include a clip or other feature to facilitate releasable coupling of control lever 232 to control wheel 230. Although a T-shaped body is described above, in other embodiments, additional or alternative configurations may be used, such as a generally cylindrical or bulbous knob, etc.

To facilitate effective control and routing of pull wires 208 within inner cavity 204 of handle 110, compression coil stop 234 may be configured to receive pull wires 208 at its forward end and guide and support pull wires 208 toward its rearward end in a manner which avoids interaction between pull wire 208a and pull wire 208b. In some embodiments, pull wires 208 includes Bowden-style cables having an inner wire 300 an outer compression coil (which is an incompressible spring) 302, as shown in FIG. 3C and 3D. Compression coils 302 extends between compression coil stop 234 and flexible tip 132 and inner wires 300 extends between control wheel 230 and flexible tip 132.

During operation, when pull wires 208 are actuated either forward or backward, corresponding pull wire tension increases to enable articulation, and a resultant compressive force must be transferred back to handle 110. This force transfer is accomplished by the respective compression coil 302 taking the load and transferring it back to the handle 110 via compression coil stop 234. Without compression coil stop 234, this load would travel thru intermediate and proximal portions 128/130 of shaft 112 and may result in shaft 112 moving in an uncontrolled and or undesirable manner when tip 132 is articulated. Upon assembly, compression coils 302 are secured within a forward portion of compression coil stop 234, effectively fixing compression coils 302 to handle 110 and allowing inner wires 300 to slide therethrough.

In some embodiments, compression coil stop 234 includes a generally X-shaped body 276 having a forward end 278 and a rearward end 280. X-shaped body 276 includes a first groove 282 extending between forward end 278 and rearward end 280 for receiving pull wire 208a and a second groove 284 for receiving pull wire 208b extending between forward end 278 and rearward end 280 in a manner which does not conflict with first groove 282. For example, first groove 282 may be formed on an edge of X-shaped body 276 and includes a first angled support surface 286 through a center of X-shaped body 276. Second groove 284 may be formed on a top of X-shaped body 276 and includes a second angled support surface 288 formed on a top of X-shaped body 276. Compression coils 302 of pull wire 208a is captured in a portion of first groove 282, while compression coils 302 of pull wire 208b is captured in a portion of second groove 284. In this configuration inner wire 300 of pull wire 208a and inner wire 300 of pull wire 208b are allowed to non-interferingly cross each other to provide smooth operation of flexible tip control mechanism 116 without requiring additional routing supports within handle 110.

Turning now to working channel 290, in some embodiments, working channel 290 and its related components are configured for insertion between right shell 200 and left shell 202 and within shaft 112 during assembly. The working channel 290 components operate to couple one or more external devices, such as irrigation fluid source 108, a surgical instrument, a source of negative pressure, etc. to and possible through flexible tip 132.

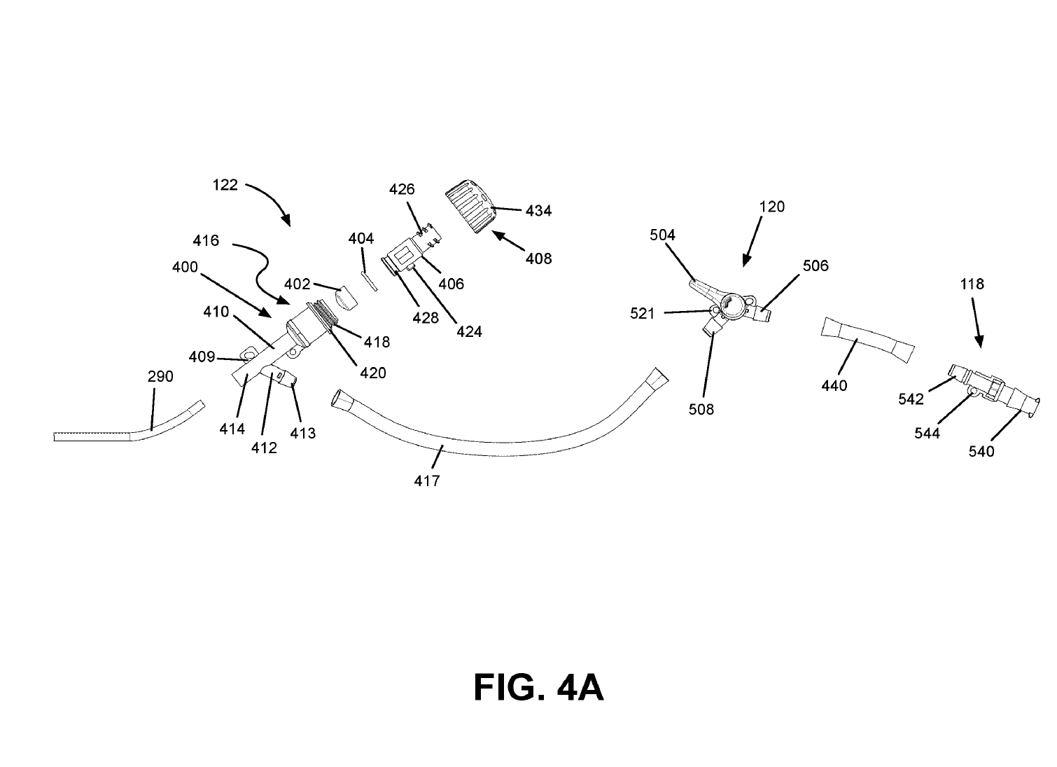

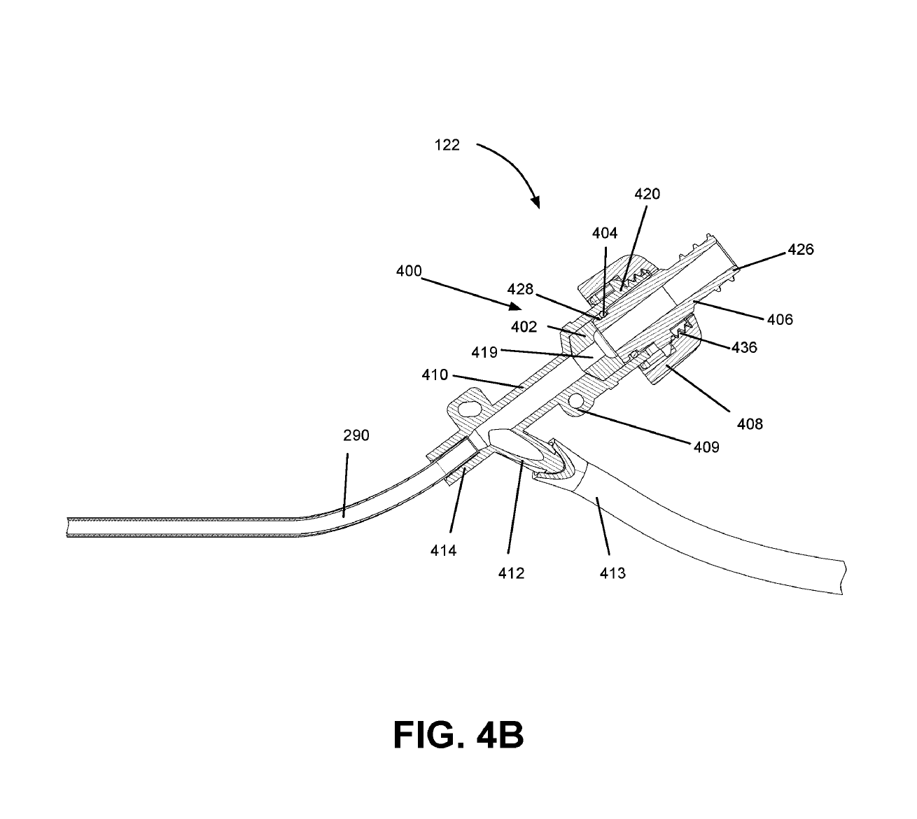

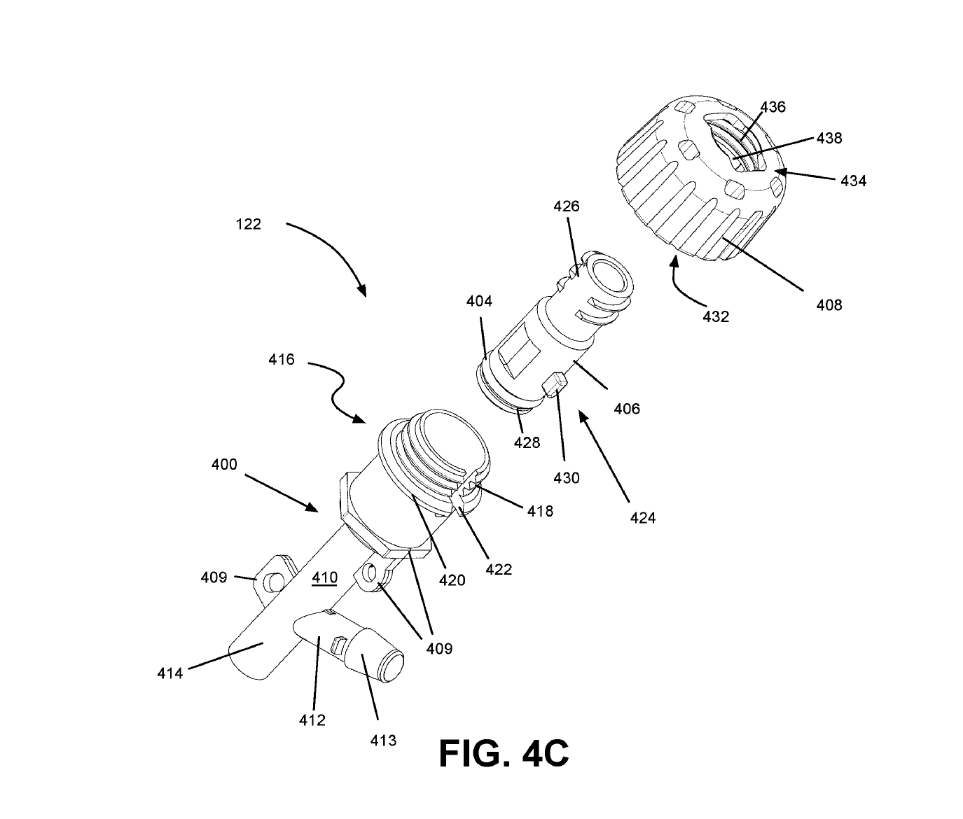

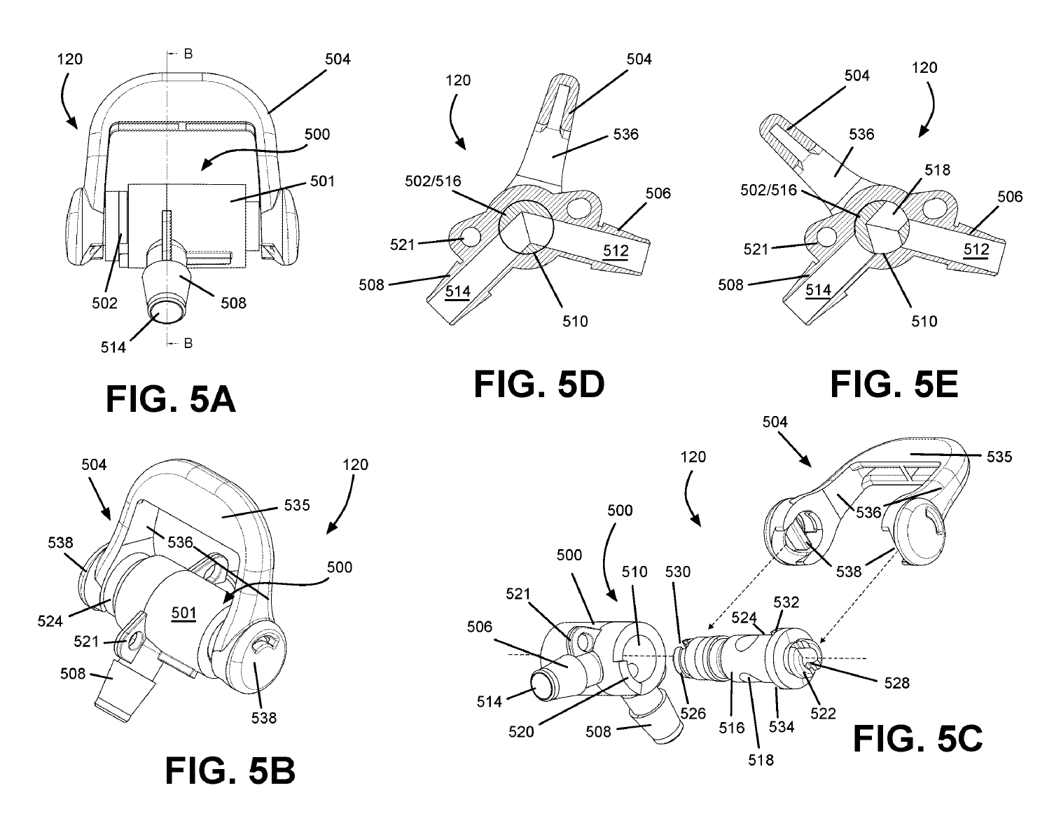

FIG. 4A is an exploded left side view of access port assembly 122, irrigation control assembly 120, and irrigation port 118, FIG. 4B is a cross-sectional view of access port assembly 122, and FIG. 4C is an exploded rear isometric view of access port assembly 122, in accordance with some embodiments. FIGS. 5A-5C are front, front left isometric, and exploded rear left isometric of irrigation control assembly 120, FIG. 5D is a cross-sectional view of irrigation control assembly 120 taken along the line B-B in FIGS. 5A and 5E, is a cross-sectional view of irrigation control assembly 120 in a closed configuration, in accordance with some embodiments.

In some embodiments, access port assembly 122, irrigation control assembly 120, and irrigation port 118 are configured to provide controlled access to working channel 290 for both irrigation fluid and other possible devices, medications, etc. In some embodiments, access port assembly 122 includes a tube fitting housing 400, compression seal 402, O-ring seal 404, accessory fitting 406, and collar 408. Tube fitting housing 400 is a generally tubular structure formed of a rigid or semi-rigid material and includes one or more fixation features 409 that correspond to engagement structures provided in right and left shells 200/202. For example, as shown in FIG. 2B, tube fitting housing 400 includes a number of attachment flanges/apertures configured to align with projections in right and/or left shells 200/202.

In some embodiments, tube fitting housing 400 includes an accessory inlet 410, an irrigation inlet 412, and a working channel outlet 414. Accessory inlet 410 is configured to provide external access to working channel 290 via accessory fitting 406 and seals 402/404. As shown in FIG. 4C, accessory inlet 410 includes a fitting interface portion 416 configured to receive accessory fitting 406 therein. Consistent with embodiments described herein, fitting interface portion 416 includes an outer threaded surface 418 and a shoulder portion 420 rearward of outer threaded surface 418. Further, as shown in FIG. 4C, fitting interface portion 416 further includes a positioning slot 422 for positively engaging a corresponding alignment feature in accessory fitting 406, as described below.

In some embodiments, irrigation inlet 412 is configured to receive an irrigation connector tube 417 (FIGS. 2A and 2B) that is coupled to irrigation control assembly 120, which is described in detail below in relation to FIGS. 5A-5E. In some embodiments, for example as illustrated in FIGS. 4A and 4C, irrigation inlet 412 may include a barbed tip 413 for securely receiving irrigation connector tube 417. Working channel outlet 414 is oriented and sized to receive a proximal end of working channel 290.

In some embodiments, compression seal 402 is formed of a resilient material and includes an aperture 419 aligned with accessory inlet 410. Compression seal 402 may comprise or include a hemostasis valve that includes a slit or other self-sealing configuration, such that withdrawal of fluid from inlet 410 is provided by forceful drawing of suction through seal 402, which prevents unintended fluid leakage prior to or during use. Upon insertion of accessory fitting 406 into accessory inlet 410, and rotation of collar 408 onto threaded outer surface 418 of fitting interface portion 416, compression seal 402 may be compressed to form a fluid-tight seal between accessory fitting 406 and tube fitting housing 400. As described below in relation to accessory fitting 406, in some embodiments, O-ring seal 404 is configured to provide a secondary seal between accessory fitting 406 and tube fitting housing 400.

In some embodiments, suitable accessory fittings 406 are configured to securely engage accessory inlet 410. A suitable accessory fitting 406 may include a female luer lock fitting or similar device. Accessory fitting 406 may include a generally tubular device configured to engage external accessory devices, such as male luer lock syringes, etc. In this manner, accessory fitting 406 may include an accessory port engaging portion 424 and an accessory device engaging portion 426. As shown in FIG. 4C, accessory port engaging portion 424 may include a generally tubular shape having an O-ring notch 428 and an alignment feature 430.

In some embodiments, collar 408 includes a tubular configuration having an open end 432, closed end 434, and a threaded inside surface 436. Closed end 434 includes a central aperture 438 configured to allow accessory device engaging portion 426 to project therethrough. In some embodiments, central aperture 438 is smaller than that of accessory port engaging portion 424 to thereby prevent accessory fitting 406 from passing completely therethrough. Threaded inside surface 436 of collar 408 may engage threaded surface 418 of fitting interface portion 416 to effect secure and sealed installation of accessory fitting within accessory inlet 410.

During installation of accessory fitting 406 within accessory inlet 410, O-ring seal 404 may be positioned within O-ring notch 428 and accessory portion engaging portion 424 may be inserted into accessory inlet 410. If necessary, alignment feature 430 may be aligned with positioning slot 422 to allow accessory fitting 406 to slide within accessory inlet 410. Collar 408 may be tightened onto accessory inlet 410. This functionality, for example, enables connecting a syringe into the access port assembly 122 in a leak free manner, so that fluids can be subsequently administered or withdrawn to/from working channel 290 without leakage.

In some embodiments, irrigation control assembly 120 includes an irrigation control valve 500 having a valve body 501, a valve insert 502, and an irrigation control lever 504. Valve body 501 may include a generally tubular body having an irrigation valve inlet 506 and an irrigation valve outlet 508 projecting outwardly therefrom. For example, valve inlet 506 may project rearwardly from valve body 501 and valve outlet 508 may project forwardly from valve body 501. In some embodiments, irrigation valve inlet 506 and irrigation valve outlet 508 are angled with respect to each other to accommodate packaging and ergonomics with respect to placement of irrigation control assembly 120 within handle 110 for efficient and comfortable control of the delivery of irrigation fluid during use of scope device 102. For example, irrigation valve inlet 506 and irrigation valve outlet 508 may be angled approximately 120º with respect to each other.

Furthermore, as shown in FIG. 5A, in one embodiment, each of valve inlet 506 and valve outlet 508 may be offset with respect to a midline of valve body 501. Such a configuration allows irrigation port tube 440 and irrigation connector tube 417 to be positioned adjacent right shell 200 within inner cavity 204, and rightwardly relative to control wheel 230 and pull wires 208. In some embodiments, each of irrigation valve inlet 506 and outlet 508 may include a barbed configuration to allow secure connection to irrigation port tube 440 and irrigation connector tube 417.

In some embodiments, valve body 501 includes a transverse aperture 510 configured to receive valve insert 502. Transverse aperture 510 is open to the passageways 512 and 514 in irrigation inlet 506 and irrigation outlet 508, respectively. As shown in FIG. 5C, valve body 501 further includes a stop portion 520 configured to interact with shoulder stop portion 524 in valve insert 502 to limit a range of movement of valve insert 502 within transverse aperture 510. As shown, stop portion 520 includes a partially notched end surface of valve body that positively defines a range through which valve insert 502 may rotate, as described below. As shown in FIG. 5B, irrigation valve body 501 may include one or more fixation features 521 that facilitate secure placement of valve body 501 within handle 110.

In some embodiments, valve insert 502 includes a generally cylindrical body 516 configured to be rotatably received within transverse aperture 510 of valve body 501. Valve insert 502 includes a valve aperture 518 therethrough that is aligned with passageways 512 and 514 when valve insert 502 is in a fully open position, as shown in FIG. 5D. Accordingly, configuration of valve aperture 518 substantially matches the angled configuration of passageways 512 and 514 in irrigation inlet 506 and outlet 508 (e.g., approx. 120º). When valve insert 502 is rotated to a fully closed position, as shown in FIG. 5E, valve aperture 518 is fully misaligned with passageways 512 and 514 to prevent flow of irrigation fluid.

In some embodiments, valve insert 502 also includes a first irrigation control level engaging portion 522 on a first end of cylindrical valve insert 502, a shoulder stop portion 524 adjacent first irrigation control level engaging portion 522, and a second irrigation control lever engaging portion 526 on a second end of cylindrical valve insert 502. As shown in FIGS. 1 and 2B, in one embodiment, each of right shell 200 and left shell 202 are provided with openings 291 that allow first and second irrigation control level engaging portion 522/526 to project outside of handle 110. Once installed within handle 110, first and second irrigation control level engaging portion 522/526 may be coupled to irrigation control lever 504 in the manner described below.

In some embodiments, each of first and second irrigation control level engaging portion 522/526 are configured to securely engage portions of irrigation control lever 504 upon assembly. For example, first and second irrigation control level engaging portions 522, 526 may include slots 528, 530, respectively, for engaging corresponding projections in irrigation control lever 504, as described below. Alternatively, different engagement features may be provided to secure valve insert 502 to irrigation control lever 504 in a manner that enables direct rotation of valve insert upon longitudinal movement of irrigation control lever 504, such as clips, fasteners, adhesives, etc.

In some embodiments, shoulder stop portion 524 of valve insert 502 includes an annular projection 532 that has an outside diameter larger than that of transverse aperture 510. As shown in FIG. 5C, a notch portion 534 of annular projection 532 includes a partial annular notch configured to interact and engage with stop portion 520 of valve body 501. In one embodiment, stop portion 520 defines an approximately 60º range through which insert may not rotate. The remaining 300º accommodates passage of shoulder stop portion 524, to allow valve insert 502 to move through a range between fully open and fully closed.

Irrigation control lever 504 may include a generally U-shaped structure having a cross member 535 and a pair of legs 536 that project perpendicularly from cross member 535 and parallel to each other. As briefly described above, legs 536 include irrigation valve insert engaging portions 538 at their distal ends to engage first and second irrigation control level engaging portions 522/526. For example, irrigation valve insert engaging portions 538 may include clips for securing legs 536 to respective ends of valve insert 502. In one embodiment, during installation, cross member 535 and/or legs 536 may flex to allow irrigation valve insert engaging portions 538 to clip onto first and second irrigation control level engaging portions 522/526.

In some embodiments, irrigation control assembly 120 is positioned on handle 110 opposite control lever 232, so as to allow ergonomic and potentially simultaneous use of both control lever 232 and irrigation control lever 504. During use, irrigation control lever 504 may be manually manipulated to precisely adjust the flow of irrigation fluid into working channel 290. For example, a user may manually manipulate control lever 504 using their index finger while gripping handle 110 at grip portion 114 and manipulating control lever 232 with their thumb. Such a control mechanism allows efficient one-handed, targeted manipulation of both flexible tip 132 and irrigation flow via control lever 232 and irrigation control lever 504, respectively.

In some embodiments, irrigation inlet 506 in valve body 501 is coupled to irrigation port 118 via irrigation port tube 440. Consistent with embodiments described herein, irrigation port 118 may include a one-piece component that includes an inlet 540 for coupling to external irrigation fluid source 108 and an outlet 542 for coupling to an inlet side of irrigation port tube 507. As shown in FIG. 4A, irrigation port 118 may include one or more fixation features 544 that facilitate secure placement of irrigation port 118 within handle 110. In one embodiment, an inlet side of irrigation port 118 may include a female luer fitting. As shown, consistent with described embodiments, irrigation port 118 is provided at a proximal end of handle 110 to allow for coupling to fluid source 108 in a manner that does not inhibit use of medical scope device 102.

Various embodiments of endoscopic devices are disclosed herein. In a first embodiment, a medical scope device includes a handle; a shaft projecting from the handle, wherein the shaft includes a proximal end coupled to the handle and a distal end; a working channel extending through the shaft; and a flexible tip coupled to the distal end of the shaft, wherein the handle comprises: a flexible tip control mechanism operatively coupled to the flexible tip; a control lever coupled to the flexible tip control mechanism, wherein the control lever extends from the handle in a first location, wherein manipulation of the control lever causes deflection of the flexible tip; and an irrigation control system for controlling a flow of irrigation fluid from an irrigation fluid source to the working channel, wherein the irrigation control system includes an irrigation control lever that extends from the handle in a second location, and wherein the first location is substantially opposite to the second location.

In a second embodiment, the medical scope device of the first embodiment, wherein the irrigation control system further comprises an irrigation control valve having a valve body and a valve insert, wherein the valve body includes a transverse aperture extending therethrough, wherein the valve insert is configured for rotational receipt within the transverse aperture in the valve body, and wherein the irrigation control lever is operatively coupled to the valve insert.

In a third embodiment, the medical scope device of the second embodiment, wherein the valve body comprises a valve inlet and a valve outlet projecting outwardly therefrom, and wherein the valve inlet and valve outlet are angled with respect to each other.

In a fourth embodiment, the medical scope device of the third embodiment, wherein the valve inlet and valve outlet are angled approximately 120º relative to each other.

In a fifth embodiment, the medical scope device of the third or fourth embodiment, wherein the valve body comprises a midline, and wherein the valve inlet and the valve outlet are offset with respect to the midline.

In a sixth embodiment, the medical scope device of the third through fifth embodiment, wherein the valve insert comprises: a generally cylindrical body having a valve aperture extending therethrough configured to align with passageways in the valve inlet and the valve outlet when the valve insert is in a fully open position; and at least one lever engaging portion for engaging at least a portion of the irrigation control lever, such that movement of the irrigation control lever causes rotation of the valve insert within the valve body.

In a seventh embodiment, the medical scope device of the sixth embodiment, wherein the cylindrical body includes a first end and a second end, wherein the first end includes a first lever engaging portion and the second end includes a second lever engaging portion.

In an eight embodiment, the medical scope device of the seventh embodiment, wherein the valve body further comprises a first end and a second end, wherein the first end includes a stop portion that includes a partially notched end surface, and wherein the first end of the valve insert comprises a shoulder stop portion configured to engage with the stop portion in the first end of the valve body to define a range through which the valve insert may rotate within the valve body.

In a ninth embodiment, the medical scope device of the seventh or eighth embodiment, wherein the handle comprises openings through which first lever engaging portion and second lever engaging portion project.

In a tenth embodiment, the medical scope device of the seventh through ninth embodiment, wherein the irrigation control lever includes: a cross member; a first leg; and a second leg, wherein the first leg and the second leg project downwardly from the cross member, and wherein distal ends of the first leg and the second leg securely engage the first lever engaging portion and the second lever engaging portion of the valve insert, respectively.

In an eleventh embodiment, the medical scope device of the tenth embodiment, wherein the distal ends of the first leg and the second leg comprises clips for engaging the first lever engaging portion and the second lever engaging portion of valve insert, respectively.

In a twelfth embodiment, the medical scope device of any of the first through eleventh embodiment, further including an irrigation fluid port that projects rearwardly from the handle, wherein the irrigation fluid port is coupled to the irrigation control system within the handle.

In a thirteenth embodiment, the medical scope device of any of the first through twelfth embodiment, wherein the handle includes a grip portion positioned forwardly of the first location and the second location.

In a fourteenth embodiment, the medical scope device of the thirteenth embodiment wherein the handle further comprises an access port assembly for allowing external access to the working channel, wherein the access port assembly is positioned forwardly of the grip portion at a third location, wherein the access port assembly comprises a tube fitting housing that includes an accessory inlet, an irrigation inlet, and a working channel outlet, wherein the irrigation inlet is coupled to the irrigation control system within the housing, and wherein the working channel outlet is coupled to the working channel.

In a fifteenth embodiment, the medical scope device of the fourteenth embodiment, further including an imaging module located in the flexible tip, wherein the handle further comprises a data interface that provides an interface between the imaging module and an external video monitor, and wherein the data interface is positioned forwardly of the grip portion at a fourth location substantially opposite to the third location of the access port assembly.

In a sixteenth embodiment, a cystoscope device including a handle; a shaft projecting from the handle, wherein the shaft includes a flexible tip; and a working channel extending through the shaft, wherein the handle comprises: a flexible tip control mechanism operatively coupled to the flexible tip, wherein the flexible tip control mechanism includes a flexible tip control lever that projects from the handle at a first location; an irrigation fluid port for coupling to an irrigation fluid source; and an irrigation control system for controlling a flow of irrigation fluid from the irrigation fluid port to the working channel, wherein the irrigation control system includes an irrigation control lever that projects from the handle at a second location, and wherein the first location is substantially opposite to the second location.

In a seventeenth embodiment, the cystoscope device of the sixteenth embodiment, wherein the handle comprises a grip portion positioned forwardly of both the first location and the second location.

In an eighteenth embodiment, the cystoscope device of the seventeenth embodiment, wherein the flexible tip control lever is configured to be manipulated by a user’s thumb and wherein the irrigation control lever is configured to be manipulated by a user’s finger.

In a nineteenth embodiment, the cystoscope device of the seventeenth or eighteenth embodiment, wherein the handle further comprises an access port assembly for allowing external access to the working channel, wherein the access port assembly is positioned forwardly of the grip portion at a third location.

In a twentieth embodiment, the cystoscope device of the nineteenth embodiment, further including an imaging module located in the flexible tip, wherein the handle further comprises a data interface that provides an interface between the imaging module and an external video monitor, and wherein the data interface is positioned forwardly of the grip portion at a fourth location substantially opposite to the third location of the access port assembly.

In a twenty-first embodiment, a method of using the medical scope device of any of the first through fifteenth embodiments.

In a twenty-second embodiment, a method of using the cystoscope of any of the sixteenth through twentieth embodiments.

In a twenty-third embodiment, a kit including the medical scope device of any of the first through fifteenth embodiments.

In a twenty-fourth embodiment, a kit including the cystoscope of any of the sixteenth through twentieth embodiments.

The foregoing description of embodiments provides illustration but is not intended to be exhaustive or to limit the embodiments to the precise form disclosed. In the preceding description, various embodiments have been described with reference to the accompanying drawings. However, various modifications and changes may be made thereto, and additional embodiments may be implemented, without departing from the broader scope of the invention as set forth in the claims that follow. The description and drawings are accordingly to be regarded as illustrative rather than restrictive.

As set forth in this description and illustrated by the drawings, reference is made to “an embodiment,” “embodiments,” etc., which may include a feature, structure, or characteristic in connection with an embodiment(s). However, the use of the phrase or term “an embodiment,” “embodiments,” etc., in various places in the specification does not necessarily refer to all embodiments described, nor does it necessarily refer to the same embodiment, nor are separate or alternative embodiments necessarily mutually exclusive of other embodiment(s). The same applies to the term “embodiment,” “embodiments,” etc.

The terms “a,” “an,” and “the” are intended to be interpreted to include one or more items. Further, the phrase “based on” is intended to be interpreted as “based, at least in part, on,” unless explicitly stated otherwise. The term “and/or” is intended to be interpreted to include any and all combinations of one or more of the associated items.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements. Similarly, relative terms, such as right, left, forward, rearward, top, bottom, etc. are used to describe relative locations and positioning of various elements described herein and do not constitute absolute or permanent directions or positions relative to an external perspective.

No element, act, or instruction described in the present application should be construed as critical or essential to the embodiments described herein unless explicitly described as such.

What is claimed is:

1. A medical scope device, comprising:
a handle;
a shaft projecting from the handle, wherein the shaft includes a proximal end coupled to the handle and a distal end;
a working channel extending through the shaft; and
a flexible tip coupled to the distal end of the shaft,
wherein the handle comprises:
a flexible tip control mechanism operatively coupled to the flexible tip;
a control lever coupled to the flexible tip control mechanism, wherein the control lever extends from the handle in a first location, wherein manipulation of the control lever causes deflection of the flexible tip; and
an irrigation control system for controlling a flow of irrigation fluid from an irrigation fluid source to the working channel, wherein the irrigation control system includes an irrigation control lever that extends from the handle in a second location, and wherein the first location is substantially opposite to the second location.

2. The medical scope device of claim 1, wherein the irrigation control system further comprises an irrigation control valve having a valve body and a valve insert, wherein the valve body includes a transverse aperture extending therethrough, wherein the valve insert is configured for rotational receipt within the transverse aperture in the valve body, and wherein the irrigation control lever is operatively coupled to the valve insert.

3. The medical scope device of claim 2, wherein the valve body comprises a valve inlet and a valve outlet projecting outwardly therefrom, and wherein the valve inlet and valve outlet are angled with respect to each other.

4. The medical scope device of claim 3, wherein the valve inlet and valve outlet are angled approximately 120º relative to each other.

5. The medical scope device of claim 3, wherein the valve body comprises a midline, and wherein the valve inlet and the valve outlet are offset with respect to the midline.

6. The medical scope device of claim 3, wherein the valve insert comprises:
a generally cylindrical body having a valve aperture extending therethrough configured to align with passageways in the valve inlet and the valve outlet when the valve insert is in a fully open position; and
at least one lever engaging portion for engaging at least a portion of the irrigation control lever, such that movement of the irrigation control lever causes rotation of the valve insert within the valve body.

7. The medical scope device of claim 6, wherein the cylindrical body includes a first end and a second end, wherein the first end includes a first lever engaging portion and the second end includes a second lever engaging portion.

8. The medical scope device of claim 7, wherein the valve body further comprises a first end and a second end, wherein the first end includes a stop portion that includes a partially notched end surface, and wherein the first end of the valve insert comprises a shoulder stop portion configured to engage with the stop portion in the first end of the valve body to define a range through which the valve insert may rotate within the valve body.

9. The medical scope device of claim 7, wherein the handle comprises openings through which first lever engaging portion and second lever engaging portion project.

10. The medical scope device of claim 7, wherein the irrigation control lever comprises:
a cross member;
a first leg; and
a second leg, wherein the first leg and the second leg project downwardly from the cross member, and wherein distal ends of the first leg and the second leg securely engage the first lever engaging portion and the second lever engaging portion of the valve insert, respectively.

11. The medical scope device of claim 10, wherein the distal ends of the first leg and the second leg comprises clips for engaging the first lever engaging portion and the second lever engaging portion of valve insert, respectively.

12. The medical scope device of claim 1, further comprising an irrigation fluid port that projects rearwardly from the handle, wherein the irrigation fluid port is coupled to the irrigation control system within the handle.

13. The medical scope device of claim 1, wherein the handle includes a grip portion positioned forwardly of the first location and the second location.

14. The medical scope device of claim 13, wherein the handle further comprises an access port assembly for allowing external access to the working channel, wherein the access port assembly is positioned forwardly of the grip portion at a third location, wherein the access port assembly comprises a tube fitting housing that includes an accessory inlet, an irrigation inlet, and a working channel outlet, wherein the irrigation inlet is coupled to the irrigation control system within the housing, and wherein the working channel outlet is coupled to the working channel.

15. The medical scope device of claim 14, further comprising an imaging module located in the flexible tip, wherein the handle further comprises a data interface that provides an interface between the imaging module and an external video monitor, and wherein the data interface is positioned forwardly of the grip portion at a fourth location substantially opposite to the third location of the access port assembly.

16. A cystoscope device, comprising:
a handle;
a shaft projecting from the handle, wherein the shaft includes a flexible tip; and
a working channel extending through the shaft, wherein the handle comprises:
a flexible tip control mechanism operatively coupled to the flexible tip, wherein the flexible tip control mechanism includes a flexible tip control lever that projects from the handle at a first location;
an irrigation fluid port for coupling to an irrigation fluid source; and
an irrigation control system for controlling a flow of irrigation fluid from the irrigation fluid port to the working channel, wherein the irrigation control system includes an irrigation control lever that projects from the handle at a second location, and wherein the first location is substantially opposite to the second location.

17. The cystoscope device of claim 16, wherein the handle comprises a grip portion positioned forwardly of both the first location and the second location.

18. The cystoscope device of claim 17, wherein the flexible tip control lever is configured to be manipulated by a user’s thumb and wherein the irrigation control lever is configured to be manipulated by a user’s finger.

19. The cystoscope device of claim 17, wherein the handle further comprises an access port assembly for allowing external access to the working channel, wherein the access port assembly is positioned forwardly of the grip portion at a third location.

20. The cystoscope device of claim 19, further comprising an imaging module located in the flexible tip, wherein the handle further comprises a data interface that provides an interface between the imaging module and an external video monitor, and wherein the data interface is positioned forwardly of the grip portion at a fourth location substantially opposite to the third location of the access port assembly.
